(19) United States
(12) Patent Application Publication
(10) Pub. No.: US 2006/0269617 A1
(43) Pub. Date: Nov. 30, 2006

(54) Supplement compositions and method of use for enhancement of insulin sensitivity

(75) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(73) Assignee: Suracell, Inc., Montclair, NJ (US)

(21) Appl. No.: 11/438,204

(22) Filed: May 22, 2006

Related U.S. Application Data
(60) Provisional application No. 60/685,141, filed on May 26, 2005.

(51) Int. Cl. A61K 31/555; A61K 31/51; A61K 31/385; A61K 33/26; A61K 33/24; A61K 31/198

(52) U.S. Cl. 424/646; 424/655; 514/440; 514/276; 514/565; 514/184

(57) ABSTRACT

A dietary supplement composition for enhancement of the insulin sensitivity is provided, which includes thiamin, chromium, alpha-lipoic acid, L-carnosine, and vanadium. The supplement composition further includes cinnamon bark and banana leaf extract (corosolic acid) for an extra strength formula. Alternatively, the supplement composition includes a herbal blend which includes boswellic acid, gymnema sylvestre leaf extract, and bitter melon extract. Further provided is a method of using the supplement composition for enhancing an individual's insulin sensitivity.

### CROSS REFERENCE TO RELATED APPLICATION

This application claims the benefit under 35 USC 119 (e) of the provisional patent application Ser. No. 60/685,141, filed May 26, 2005, which is herein incorporated by reference in its entirety.

### FIELD OF THE INVENTION

The present invention relates to dietary supplement compositions and the method of use thereof for enhancing insulin sensitivity of a person.

### BACKGROUND OF THE INVENTION

Insulin is one of the most powerful anabolic hormones in the body and is the primary driver of amino acid and glucose into muscle cells. Insulin also has a number of other important physiological effects, including increased synthesis and retention of protein in skeletal muscle and other tissues; stimulation of activated immune cells; enhanced brain uptake of tyrosine and tryptophan (precursors for important brain neurotransmitters); reduced output of free fatty acids from adipose stores; accelerated potassium uptake by cells; and increased metabolic rate.

Insulin resistance is associated with major public health problems such as diabetes, obesity and cardiovascular diseases. Studies have indicated a tendency for mature adults to lose sensitivity to insulin. It has been found that sedentary lifestyle, obesity, a diet low in fiber and chromium, and high in sugars, all contribute to decreased insulin sensitivity. Studies have found that individuals with impaired insulin sensitivity are at risk for high blood pressure, heart disease, and diabetes.

Glycation is the result of a sugar molecule, such as fructose or glucose, bonding to a protein or lipid molecule without the controlling action of an enzyme. Over time, the sugar moieties bound to the glycated proteins are chemically modified to become molecular structures called Advanced Glycation Endproducts (A.G.E.s). A.G.E.s can interfere witn the proper functioning of the proteins to which they are attached. Furthermore, some of the A.G.E.s form covalent crosslinks with adjacent protein strands, which stiffens tissues. Glycation and crosslinking have been implicated as strong contributors to many progressive diseases of aging, including vascular diseases, erectile dysfunction, kidney disease, stiffness of joints and skin, arthritis, cataracts, retinopathy, neuropathy, Alzheimer's Dementia, impaired wound healing, urinary incontinence, complications of diabetes, and cardiomyopathies. It is known that insulin sensitivity and glycation are closely related to each other. Increase in insulin sensitivity reduces glycation.

It has been reported that vanadium and chromium, when ingested, have properties that closely mimic many of the physiological effects of insulin. In this respect, it has been found that these elements serve to both increase the effectiveness and enhance the anabolic effects of insulin. Supplementation of these elements into a normal diet has been shown to increase lean body mass without increasing body fat, stabilize blood sugar, increase the responsiveness of cells to insulin, and lower blood fat levels. By their ability to potentiate the effect of insulin, both vanadium and chromium have been found to enhance the entry of glucose (for energy) and amino acids (for protein synthesis) into muscle cells and inhibit the action of enzymes that catabolize the amino acids and proteins. It has further been found that the results of supplementing these elements include cholesterol lowering, energy producing, and anabolic promoting properties, while providing an optimal environment for anabolic development, weight/fat loss, and energy output.

Thiamin is important to the biosynthesis of keto-acids. It is involved in transketolase reactions. Thiamin also plays a key role in the body's metabolic cycle for generating energy; and aids in the digestion of carbohydrates. Tiamin is essential for the normal functioning of the nervous system, muscles and heart; it stabilizes the appetite; and promotes growth and good muscle tone.

Cinnamon bark is widely used as a spice. The active ingredient in cinnamon is a water-soluble polyphenol compound, methylhydroxychalcone polymer (MHCP). It has been found in vitro study that MHCP mimics insulin, activates its receptor, and works synergistically with insulin in cells. It has been reported in a recent human subject study that cinnamon significantly reduces blood sugar levels in diabetes type II patients.

The active component of the commercially available banana leaf extract is corosolic acid. Corosolic acid, also known as is colosolic acid, 2 alpha-hydroxyursolic acid, banana or botanical insulin, is a triterpene compound extracted from the leaves of the plant Lagerstroemia speciosa. The leaves of Lagerstroemia speciosa are used in Southeast Asia as an herbal remedy for a number of disorders, including diabetes and obesity. In the Phillipines, the plant is known by the Tagalog name of banaba.

L-camosine is a peptide. It naturally occurs in high concentrations in skeletal muscle tissue. L-Carnosine is also an antioxidant that stabilizes cellular membranes, protecting them from damage by free radicals; therefore, it has been used as an antioxidant in dermatological products for protection of skin from UV radiation.

U.S. Pat. No. 6,572,897 (to Gorsek) teaches a blood sugar maintenance composition which comprises alpha lipoic acid, chromium, lutein, bioflavonoids (quercetin and rutin), mormordica charantia extract, corosolic acid, and gymnema sylvestre extract, and other ingredients

U.S. Pat. No. 6,528,502 (to Krumhar et al.) teaches a nutritional supplement and method of use for improved carbohydrate management. The nutritional supplement contains niacin, chromium, vanadium, and optionally thiamin, riboflavin, magnesium, and zinc.

It is desirable to provide improved supplement compositions which stimulate and enhance an individual's insulin sensitivity, particularly those with impaired insulin sensitivity, due to genetic predisposition, clinical conditions, advanced aging, or poor environmental impact, to reduce the risk of developing related clinical conditions and increase the likelihood of an individual's longevity.

### SUMMARY OF THE INVENTION

In one aspect, the present invention is directed to a supplement composition for enhancing an individual's insulin sensitivity. In one embodiment, the supplement composition comprises effective amounts of thiamin, chromium, alpha-lipoic acid, L-carnosine, and vanadium. In a further embodiment, the supplement composition further comprises effective amounts of cinnamon bark and banana leaf extract (corosolic acid) for an extra strength formula. Alternatively, the supplement composition further comprises an effective amount of a herbal blend which includes boswellic acid, gymnema sylvestre leaf extract, and bitter melon extract. Moreover, the supplement composition also comprises pharmaceutically acceptable excipients, and can be provided in a form for oral administration.

In a further embodiment, the present invention is directed to a method for enhancing an individual's insulin sensitivity, and reducing the likelihood of an individual in developing clinical conditions related to deficiency in insulin sensitivity and glycation process. In one embodiment, the method comprises orally administering a supplement composition daily in a dosage comprising from about 50 mg to about 400 mg of thiamin mononitrate, from about 100 μg to about 400 μg of chromium picolinate, from about 125 mg to about 500 mg of alpha-lipoic acid, from about 125 mg to about 500 mg of L-carnosine, and from about 25 μg to about 100 μg vanadium of vanadyl sulfate.

In another embodiment, the method comprises orally administering a supplement composition daily in a dosage comprising from about 50 mg to about 400 mg of thiamin mononitrate, from about 100 μg to about 400 μg of chromium picolinate, from about 125 mg to about 500 mg of alpha-lipoic acid, from about 125 mg to about 500 mg of L-carnosine, from about 25 μg to about 100 μg vanadium of vanadyl sulfate, from about 25 mg to about 100 mg of cinnamon bark and from about 25 mg to about 100 mg of banana leaf extract containing 1% corosolic acid.

In an alternative embodiment, the method comprises orally administering a supplement composition daily in a dosage comprising from about 50 mg to about 400 mg of thiamin mononitrate, from about 100 μg to about 400 μg of chromium picolinate, from about 125 mg to about 500 mg of alpha-lipoic acid, from about 125 mg to about 500 mg of L-carnosine, from about 25 μg to about 100 μg vanadium of vanadyl sulfate, from about 25 mg to about 100 mg of a herbal blend comprising boswellic acid, gymnema sylvestre leaf extract, and bitter melon extract.

In yet a further embodiment, the method of the present invention is directed to provide a suitable supplement composition to an individual based on the individual's genetic predisposition for glycation.

### DETAILED DESCRIPTION OF THE INVENTION

In one embodiment, the present invention provides a dietary supplement composition for enhancing an individual's insulin sensitivity. The supplement composition comprises thiamin, chromium, alpha-lipoic acid, L-carnosine, and vanadium. The supplement composition further comprises pharmaceutically acceptable excipients.

As used herein, “insulin sensitivity” refers to a person's sensitivity or response to the effectiveness of insulin. As insulin sensitivity and glycation are closely related to each other; increase of insulin sensitivity in an individual reduces the glycation process. The present invention provides novel dietary supplement compositions for enhancing insulin sensitivity, and reducing glycation, particularly for those who have more likely to have deficiency in insulin sensitivity, or glycation due to genetic predisposition, clinical conditions, advanced aging, or poor environmental impact. The supplement compositions and the method of use are described hereinafter in detail.

Chromium possesses properties that mimic and enhance the effects of insulin. When enhancing the effects of insulin, chromium indirectly assists amino acid uptake by muscle, stimulates protein synthesis, and retards the rate of protein breakdown. Additionally, by promoting insulin-stimulated brain uptake of tryptophan, chromium may aid brain synthesis of serotonin, a neurotransmitter than helps control appetite and especially sugar cravings. Trivalent chromium has a strongly positive charge that impedes its movement across cell membranes. Adequate absorption of chromium occurs best when the metal is provided in chelated form, such as amino acid chelates, vitamin acid chelates, and the like. A preferred form of chromium according to the present invention is chromium picolinate. In one embodiment of the present invention, the supplement composition comprises chromium picolinate, preferably in an amount from about 100 μg to about 400 μg in one dosage. In a preferred embodiment, the composition comprises about 200 μg of chromium picolinate.

Herein, one dosage is also referred to as one serving. If the supplement composition is provided in the form of tablet, one dosage can be either one tablet, or two to three tablets. For example, 200 μg of chromium picolinate in one dosage can be provided in one tablet as shown in Supplement Composition A of Example 1, or in two tablets as shown in Supplement Composition B of Example 2. The size and number of the tablet may depend on the manufacturability, which may further depend on the properties of the components and the pharmaceutically acceptable excipients used.

Vanadium is an essential nutrient. Vanadium deficiency can lead to slow growth, defective bones, and altered lipid metabolism. Once ingested, vanadium typically is transformed into vanadate, the salt form of vanadic acid. Vanadate ions mimic most of the action of insulin in intact cell systems via a post-receptor mechanism. Vanadate, like insulin, causes phosphorylation of the insulin receptors of fat cells and thus stimulates glucose transport, activates glycogen synthase, and increases glycogen synthesis in the fat cells. The supplement composition of the present invention comprises vanadium, preferably in the form of vanadyl sulfate. In one embodiment, the supplement composition comprises vanadyl sulfate in an amount from about 25 μg to about 100 μg in one dosage. In a preferred embodiment, the supplement composition comprises about 50 μg of vanadyl sulfate in one dosage.

Alpha lipoic acid is a lipid- and water-soluble antioxidant that works synergistically with other antioxidants in the cell's mitochondria. Alpha-lipoic acid is a cofactor for several regulatory enzymes, including pyruvate dehydrogenase, and is believed to have an effect on glucose transport and utilization. In one embodiment, the supplement composition of the present invention comprises alpha-lipoic acid, preferably in an amount from about 125 mg to about 500 mg in one dosage. In one preferred embodiment, the supplement composition comprises about 250 mg of alpha-lipoic acid in one dosage.

Thiamin or thiamine is a generic term applied to all substances possessing vitamin B-1 activity, regardless of the anion attached to the molecule. Thiamin is readily absorbed in aqueous solution from both the small and large intestine, and is then carried to the liver by the portal circulation. In the liver, as well as in all living cells, it normally combines with phosphate to form cocarboxylase. It may be stored in the liver in this form or it may combine further with manganese and specific proteins to become active enzymes known as carboxylases. The supplement composition of the present invention comprises thiamin, preferably in the form of thiamin mononitrate. In one embodiment, the supplement composition comprises thiamin mononitrate, preferably in an amount from about 50 mg to about 400 mg in one dosage. In a preferred embodiment, the supplement composition comprises about 100 mg of thiamin mononitrate in one dosage, and in another preferred embodiment the supplement composition comprises about 200 mg of thiamin mononitrate in one dosage.

L-carnosine is a dipeptide, which is a combination of alanine and histidine. In one embodiment, the supplement composition of the present invention comprises L-camosine, preferably in an amount from about 125 mg to about 500 mg in one dosage. In a preferred embodiment, the supplement composition comprises about 250 mg of L-camosine in one dosage.

Example 1 shows an exemplary supplement composition containing the above described active components. This composition is considered as a regular strength formula for the purpose of enhancing insulin sensitivity.

In a further embodiment, the supplement composition of the present invention further comprises cinnamon bark and banana leaf extract. The active ingredient in cinnamon is methylhydroxychalcone polymer (MHCP). In vitro, MHCP mimics insulin, activates its receptor, and works synergistically with insulin in cells. In human subject study, cinnamon significantly reduces blood sugar levels in diabetic patients. Banana leaf extract, containing about 1% of corosolic acid, is used the supplement composition of one embodiment of the present invention. Corosolic acid, also commonly referred as colosolic acid, is a triterpene compound.

The combination of cinnamon bark and banana leaf extract (corosolic acid) with the other active components described above provides further enhancement of insulin sensitivity. In one embodiment, the supplement composition of the present invention comprises cinnamon bark, preferably in an amount from about 25 mg to about 100 mg in one dosage, and banana leaf extract (1% corosolic acid) preferably in an amount from about 25 mg to about 100 mg in one dosage. In a preferred embodiment, the supplement composition comprises about 50 mg of cinnamon bark and about 50 mg of banana leaf extract (1% corosolic acid) in one dosage.

Example 2 shows an exemplary supplement composition which comprises thiamin, chromium picolinate, alpha-lipoic acid, L-carnosine, vanadyl sulfate, cinnamon bark and banana leaf extract (corosolic acid). This composition is considered as an extra strength formula for the purpose of enhancing insulin sensitivity.

In alternative embodiment, the dietary supplement composition of the present invention further comprises a herbal blend which comprises boswellic acid, gymnema sylvestre leaf extract, and bitter melon extract.

Boswellic acids are effective anti-inflammatory and anti-arthritic agents, for osteoarthritis and rheumatoid arthritis, soft tissue rheumatism, and low back pain. They also have cholesterol and triglyceride lowering properties. Gymnema sylvestre is a woody climbing plant that grows in the tropical forests of central and southern India, and the leaves have been used in herbal medicine preparations. Gymnema has been used in India for the treatment of diabetes for over 2,000 years. Bitter melon grows in tropical areas, where it is used as a food as well as a medicine. At least three different groups of constituents in bitter melon have been reported to have hypoglycemic (blood sugar lowering) or other actions of potential benefit in diabetes mellitus. These include a mixture of steroidal saponins known as charantin, insulin-like peptides, and alkaloids.

The combination of this herbal blend with the other active components described above provides further enhancement of insulin sensitivity. In one embodiment, the supplement composition of the present invention comprises the herbal blend which contains boswellic acid, gymnema sylvestre leaf extract, and bitter melon extract, preferably in an amount from about 25 mg to about 100 mg in one dosage. In a preferred embodiment, the supplement composition comprises about 50 mg of the herbal blend in one dosage. Example 3 shows an exemplary supplement composition which comprises thiamin, chromium picolinate, alpha-lipoic acid, L-carnosine, vanadyl sulfate, and this herbal blend.

As a convenient form of dietary supplement, the supplement compositions described above are provided in the form of tablet. However, it should be understood that tablet is only one of various convenient dosage forms which can be used for the supplement composition. Other suitable forms include hard or soft-gelatin capsules, powders, or in liquid dosage forms, such as elixirs, syrups, dispersed powders or granules, emulsions, or aqueous or oily suspensions. When other dosage forms are used, the amounts of the active components in one dosage remain the same, however, the concentration of the component in different pharmaceutical media can be different.

Preferably, the supplement composition is formulated as a tablet, and as such it can contain pharmaceutically acceptable excipients, according to methods and procedures well known in the art. As used herein, “excipients” means substances that are of little or no therapeutic value, but useful in the manufacture and compounding of various pharmaceutical preparations, which form the medium of the supplement composition. These substances include coloring, flavoring, and diluting agents; emulsifying and suspending agents; ointment bases; pharmaceutical solvents; antioxidants and preservatives for the product; and miscellaneous agents. Suitable excipients are described in Remington's Pharmaceutical Sciences, Mack Publishing Company, a standard reference text in this field, which is incorporated herein by reference in its entirety.

As used herein, “tablets” are solid pharmaceutical dosage forms containing active ingredients with or without suitable diluents and prepared either by compression or molding methods well known in the art. Although tablets are most frequently discoid in shape, they may also be round, oval, oblong, cylindrical, or triangular. They may differ greatly in size and weight depending on the amount of active ingredients present and the intended method of administration. They are divided into two general classes, (1) compressed tablets, and (2) molded tablets or tablet triturates. In addition to the active ingredients, tablets contain a number of inert excipients or additives. A first group of such excipients includes those materials that help to impart satisfactory compression characteristics to the formulation, including diluents, binders, and lubricants. A second group of such excipients helps to give additional desirable physical characteristics to the finished tablet, such as disintegrators, colors, flavors, and sweetening agents. Compressed tablets can be uncoated or can be sugar coated or film coated by known techniques to mask any unpleasant taste and protect the tablet from the atmosphere, or enteric coated for selective disintegration and adsorption in the gastrointestinal tract.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and other suitable materials. As used herein, “binders” are agents used to impart cohesive qualities to the powdered material. Binders insure the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and other suitable materials. As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. As used herein, “coloring agents” are chemicals that give tablets a more pleasing appearance, and in addition help the manufacturer to control the product during its preparation and help the user to identify the product. Any of the approved certified water-soluble FD&C dyes, mixtures thereof, can be used to color tablets.

In a further aspect, the present invention provides the method of using the supplement compositions described above as a dietary supplement to individuals, particularly those in need thereof. Preferably, the supplement composition is administrated daily. The preferred daily dosage includes from about 50 to about 400 mg of thiamin, from about 100 to about 400 μg of chromium picolinate, from about 125 to about 500 mg of alpha-lipoic acid, from about 125 to about 500 mg of L-carnosine, from about 25 to about 100 μg of vanadyl sulfate. Furthermore, the preferred daily dosage further includes from about 25 mg to about 100 mg of cinnamon bark and from about 25 mg to about 100 mg of banana leaf extract (1% corosolic acid). Alternatively, the preferred daily dosage further includes from about 25 to 100 mg of the herbal blend which comprises boswellic acid, gymnema sylvestre leaf extract, and bitter melon extract.

Supplement Composition A of Example 1 is considered as a regular strength formula for enhancing an individual's insulin sensitivity. The preferred daily dosage is one tablet of Supplement Composition A, which includes about 100 mg of thiamin, about 200 μg of chromium picolinate, about 250 mg of alpha-lipoic acid, about 250 mg of L-carnosine, and about 50 μg of vanadyl sulfate. Supplement Composition B of Example 2 is considered as an extra strength formula, which is particularly suitable for individuals who have deficient insulin sensitivity due to genetic predisposition, clinical conditions, advanced aging, or poor environmental impact, and therefore, particularly in need of enhancing insulin sensitivity. For these individuals, a preferred daily dosage is two tablets of Supplement Composition B, which has a daily dosage of about 200 mg of thiamin, about 200 μg of chromium picolinate, about 250 mg of alpha-lipoic acid, about 250 mg of L-carnosine, about 50 μg of vanadyl sulfate, about 50 mg of cinnamon bark and about 50 mg of banana leaf extract (1% corosolic acid).

A method of determining an individual's genetic predisposition for deficiency in glycation, in other word more prone to have excess glycation, is described in a co-pending patent application Ser. No. 60/796,423, entitled “Method of Determining Genetic Predisposition for Deficiency in Health Functions Using SNP Analysis”, which is herein incorporated by reference in its entirety. More specifically, upon performing a SNP genotyping assay of a biological sample collected from an individual, the individual's genetic predisposition for glycation can be determined by using a specific glycation SNP panel which comprises predetermined glycation identifier SNPs. Such a SNP analysis determines and identifies an individual's genetic predisposition for glycation as normal, sub-normal, and deficient. The individuals having genetic predisposition for deficiency in glycation are more likely to develop clinical conditions directly or indirectly related to glycation.

In a further aspect, the method of the present invention provides a suitable supplement composition based on an individual's genetic predisposition for glycation. In one embodiment, an individual whose genetic predisposition for glycation is sub-normal is recommended to take one tablet of Supplement Composition A of Example 1 daily. In another embodiment of the present invention, an individual whose genetic predisposition for glycation is deficient is recommended to take two tablets of Supplement Composition B of Example 2 or Supplement Composition C of Example 3 daily. As described previously, insulin sensitivity and glycation is closely related to each other. Increase in insulin sensitivity reduces glycation. By providing a suitable supplement composition based on an individual's genetic predisposition, the method of the present invention can more effectively assist in enhancing the individual's insulin sensitivity and reducing the likelihood of the individual in developing clinical conditions related to glycation or deficient insulin sensitivity.

The following examples are illustrative of the invention and are in no way to be interpreted as limiting the scope of the invention, as defined in the claims.

### EXAMPLE 1

A composition of the following formulation was prepared in tablet form, including pharmaceutically acceptable carriers, by standard methods known to those of ordinary skill in the art:

TABLE 1
Supplement Composition A for enhancing insulin sensitivity

| | Amount Per Serving | |
|---|---|---|
| Contents | (in one tablet) | |
| Thiamin (as thiamin mononitrate) | 100 | mg |
| Chromium (as chromium picolinate) | 200 | μg |
| Alpha-lipoic acid | 250 | mg |
| L-Carnosine | 250 | mg |
| Vanadium (as vanadyl sulfate) | 50 | μg |

Other ingredients include: dicalcium phosphate, microcrystalline cellulose, croscarmellose sodium, stearic acid, magnesium stearate, silica, and pharmaceutical glaze. In the example, each tablet has a weight ranging from about 800 mg to about 1400 mg.

### EXAMPLE 2

A composition of the following formulation was prepared in tablet form, including pharmaceutically acceptable carriers, by standard methods known to those of ordinary skill in the art:

TABLE 2
Supplement Composition B for enhancing insulin sensitivity

| | Amount Per Serving | |
|---|---|---|
| Contents | (in two tablets) | |
| Thiamin (as thiamin mononitrate) | 200 | mg |
| Chromium (as chromium picolinate) | 200 | μg |
| Vanadium (as vanadyl sulfate) | 50 | μg |
| L-Carnosine | 250 | mg |
| Cinnamon bark | 50 | mg |
| Banana leaf extract (1% corosolic acid) | 50 | mg |
| Alpha-lipoic acid | 250 | mg |

Other ingredients include: dicalcium phosphate, microcrystalline cellulose, croscarmellose sodium, stearic acid, magnesium stearate, silica, and pharmaceutical glaze. In the example, each tablet has a weight ranging from about 800 mg to about 1400 mg.

### EXAMPLE 3

A composition of the following formulation was prepared in tablet form, including pharmaceutically acceptable carriers, by standard methods known to those of ordinary skill in the art:

TABLE 3
Supplement Composition C for enhancing insulin sensitivity

| | Amount Per Serving | |
|---|---|---|
| Contents | (in two tablets) | |
| Thiamin (as thiamin mononitrate) | 200 | mg |
| Chromium (as chromium picolinate) | 200 | μg |
| Vanadium (as vanadyl sulfate) | 50 | μg |
| L-Carnosine | 250 | mg |
| Herbal Blend: | 50 | mg |
| Boswellic acid, gymnema sylvestre | | |
| leaf extract, and bitter melon extract | | |
| Alpha-lipoic acid | 250 | mg |

Other ingredients include: dicalcium phosphate, microcrystalline cellulose, croscarmellose sodium, stearic acid, magnesium stearate, silica, and pharmaceutical glaze. In the example, each tablet has a weight ranging from about 800 mg to about 1400 mg.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. A supplement composition for enhancement of insulin sensitivity, comprising effective amount of:
(a) thiamin,
(b) chromium,
(c) alpha-lipoic acid,
(d) L-carnosine, and
(e) vanadium.

2. The supplement composition of claim 1, wherein said thiamin is thiamin mononitrate in amount from about 50 mg to about 400 mg in one dosage.

3. The supplement composition of claim 1, wherein said chromium is chromium picolinate in amount from about 100 μg to about 400 μg in one dosage.

4. The supplement composition of claim 1, wherein said vanadium is vanadyl sulfate in amount from about 25 μg to about 100 μg in one dosage.

5. The supplement composition of claim 1, wherein said alpha-lipoic acid is in amount from about 125 mg to about 500 mg in one dosage.

6. The supplement composition of claim 1, wherein said L-carnosine is in amount from about 125 mg to 500 mg in one dosage.

7. The supplement composition of claim 1 further comprising cinnamon bark and banana leaf extract containing 1% corosolic acid.

8. The supplement composition of claim 7, wherein said cinnamon bark is in amount from about 25 mg to about 100 mg in one dosage.

9. The supplement composition of claim 7, wherein said banana leaf extract containing 1% corosolic acid is in amount from about 25 mg to about 100 mg in one dosage.

10. The supplement composition of claim 1 further comprising a herbal blend comprising boswellic acid, gymnema sylvestre leaf extract, and bitter melon extract.

11. The supplement composition of claim 1 , wherein said composition is in a form for oral administration.

12. A supplement composition for enhancement of insulin sensitivity, comprising:
(a) from about 50 mg to about 400 mg of thiamin mononitrate;
(b) from about 100 μg to about 400 μg of chromium picolinate;
(c) from about 125 mg to about 500 mg of alpha-lipoic acid;
(d) from about 125 mg to about 500 mg of L-carnosine; and
(e) from about 25 μg to about 100 μg vanadium of vanadyl sulfate.

13. The supplement composition of claim 12 further comprising from about 25 mg to about 100 mg of a herbal blend comprising boswellic acid, gymnema sylvestre leaf extract, and bitter melon extract.

14. The supplement composition of claim 12 further comprising from about 25 mg to about 100 mg of cinnamon bark.

15. The supplement composition of claim 14 further comprising from about 25 mg to about 100 mg of banana leaf extract containing 1% corosolic acid.

16. A method for enhancing an individual's insulin sensitivity comprising orally administering a supplement composition daily in a dosage comprising from about 50 mg to about 400 mg of thiamin mononitrate, from about 100 μg to about 400 μg of chromium picolinate, from about 125 mg to about 500 mg of alpha-lipoic acid, from about 125 mg to about 500 mg of L-carnosine, and from about 25 μg to about 100 μg vanadium of vanadyl sulfate.

17. The method of claim 16, wherein said supplement composition further comprising in said dosage from about 25 mg to about 100 mg of cinnamon bark and from about 25 mg to about 100 mg of banana leaf extract containing 1% corosolic acid.

18. The method of claim 16, wherein said supplement composition further comprising in said dosage from about 25 mg to about 100 mg of a herbal blend comprising boswellic acid, gymnema sylvestre leaf extract, and bitter melon extract.
